(19) United States
(12) Patent Application Publication
(10) Pub. No.: US 2020/0016213 A1
(43) Pub. Date: Jan. 16, 2020

(54) Methods of Treating Cerebral Palsy Using High Dose Allogeneic Umbilical Cord Blood

(71) Applicant: Duke University, Durham, NC (US)

(72) Inventors: Joanne Kurtzberg, Durham, NC (US); Jessica Sun, Durham, NC (US)

(21) Appl. No.: 16/510,387

(22) Filed: Jul. 12, 2019

Related U.S. Application Data
(60) Provisional application No. 62/697,256, filed on Jul. 12, 2018.

(51) Int. Cl. A61K 35/51; A61K 9/00; A61P 21/00; A61P 25/14

(52) U.S. Cl. CPC A61K 35/51; A61P 25/14; A61P 21/00; A61K 9/0019

(57) ABSTRACT

The present disclosure relates to methods of treating cerebral palsy. More particularly, the present disclosure relates to methods of using a high dose of allogeneic umbilical cord blood to treat cerebral palsy.

### CROSS REFERENCE TO RELATED APPLICATIONS

This application claims priority to U.S. Provisional Patent Application Ser. No. 62/697,256, filed Jul. 12, 2018, the contents of which are hereby incorporated by reference in their entirety.

### FIELD OF THE INVENTION

The present disclosure relates to methods of treating cerebral palsy. More particularly, the present disclosure relates to methods of using a high dose of allogeneic umbilical cord blood to treat cerebral palsy.

### DESCRIPTION OF THE RELATED ART

Cerebral Palsy (CP) is a condition affecting young children that causes lifelong disabilities, and typically results from in utero or perinatal injury to the developing brain, such as hypoxic insult, hemorrhage, or stroke. Affected children have varying degrees of functional impairments from mild limitations in advanced motor skills to severely limited self-mobility despite use of assistive technology, resulting in a lifelong inability to function independently. Current treatments are supportive, focusing on managing sequelae with physical therapies, medications, and surgery. However, there are no curative therapies, or therapies to address the underlying brain injury.

### SUMMARY OF THE INVENTION

The present invention comprises a method of treating a patient with cerebral palsy comprising administering allogeneic cord blood at a dose of around 10×10⁷ total nucleated cells/kg. In certain embodiments of this aspect of the invention, the cord blood is administered systemically.

### DETAILED DESCRIPTION OF THE INVENTION

Before the disclosed processes and materials are described, it is to be understood that the aspects described herein are not limited to specific embodiments, apparati, or configurations, and as such can, of course, vary. It is also to be understood that the terminology used herein is for the purpose of describing particular aspects only and, unless specifically defined herein, is not intended to be limiting.

It is also to be understood that unless clearly indicated otherwise by the context, embodiments disclosed for one aspect or embodiment of the invention can be used in other aspects or embodiments of the invention as well, and/or in combination with embodiments disclosed in the same or other aspects of the invention. Thus, the disclosure is intended to include, and the invention includes, such combinations, even where such combinations have not been explicitly delineated.

### Definitions

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

As used herein, “treatment,” “therapy,” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient that has, or is suffering from, cerebral palsy or a hypoxic-ischemic brain injury.

As used herein, the term “disease” refers to any condition that is abnormal, such as a disorder or a structure or function, that affects part or all of a subject. In some embodiments, the disease comprises a neurological disorder. In certain embodiments, the neurological disorder comprises cerebral palsy; in other embodiments, the neurological disorder comprises a hypoxic-ischemic brain injury.

As used herein, the term “cerebral palsy” (CP) refers to any one of a number of neurological disorders that appear in infancy or early childhood and permanently affect body movement and muscle coordination but don't worsen over time. While cerebral palsy affects muscle movement, it isn't caused by problems in the muscles or nerves, but rather by abnormalities in parts of the brain that control muscle movements. The majority of children with cerebral palsy are born with it, or develop it as a result of a brain injury associated with the birthing process or in the neonatal period, although it may not be detected until months or years later. The early signs of cerebral palsy usually appear before a child reaches 3 years of age. The most common are a lack of muscle coordination when performing voluntary movements (ataxia); stiff or tight muscles and exaggerated reflexes (spasticity); walking with one foot or leg dragging; walking on the toes, a crouched gait, or a “scissored” gait; and muscle tone that is either too stiff or too floppy.

The inventors have determined that the administration of allogeneic umbilical cord blood cells (AlloCB) to children with cerebral palsy at a high dose confers certain benefits in bringing about improvement in motor function and brain connectivity in those patients. More particularly, these benefits are conferred at a dose of about 10×10⁷ total nucleated cells/kg. Such a high dose can be achieved through the use of banked units of allogeneic cord blood. Accordingly, the invention is directed to a method of treating a patient with cerebral palsy comprising administering allogeneic cord blood at a dose of about 10×10⁷ total nucleated cells/kg patient weight.

It is to be understood that as used herein, unless stated otherwise, the term “allogeneic cord blood” is meant to encompass allogeneic cord blood in any format and/or a component or mixture of components thereof, whether specifically so stated or not.

The use of allogeneic cord blood affords certain advantages over the use of autologous cord blood, in particular that many patients may not have autologous cord blood banked. AlloCB has been used extensively in the field of hematopoietic transplantation, has been shown to be safe, and is abundantly available.

The AlloCB may be administered at a dose of about 10×10⁷ total nucleated cells/kg patient weight. As used herein “a dose of about” means within 25% above or below the stated dose. Thus, the AlloCB may be administered at a dose between 7.5×10⁷ and 12.5×10⁷. All dosing levels falling within this range, even if not specifically recited, are to be regarded as explicitly included within the scope of the invention. Merely by way of example, the dose may be 7.5×10⁷, 8.0×10⁷, 8.5×10⁷, 9.0×10⁷, 9.5×10⁷, 10×10⁷, 10.5×10⁷, 11.0×10⁷, 11.5×10⁷, 12.0×10⁷, or 12.5×10⁷. The dose may also be within any range falling within 7.5×10⁷ to 12.5×10⁷. Any range falling within this range, even if not specifically recited, is to be regarded as explicitly included within the scope of the invention. Merely by way of example, the dose may be between 7.5×10⁷ and 10×10⁷, between 10×10⁷ and 12.5×10⁷, between 8×10⁷ and 12×10⁷, between 9×10⁷ and 11×10⁷, between 8.5×10⁷ and 11.5×10⁷, or between 7.5×10⁷ and 12.5×10⁷.

The patient may be any human or nonhuman animal. In one embodiment, the patient is human. In another embodiment, the patient is a human child under 18 years of age, or in any age range falling within this broader age range. In non-limiting examples, the patient may be a newborn, an infant 1-12 months old, 1 month to 2 years old, 1 year to 10 years old, 1 year to 8 years old, 1 year to 6 years old, 1 year to 4 years old, 1 year to 2 years old, 2 years to 10 years old, 2 years to 8 years old, 2 years to 6 years old, or 2 years to 4 years old.

The allogeneic cord blood can be preserved and prepared for administration by methods known in the art. The AlloCB may be administered to a subject by any technique known in the art, including local or systemic delivery. Routes of administration include, but are not limited to, subcutaneous, intracutaneous, intramuscular, intraperitoneal, intravenous, intrathecal, intracerebral, intraventricular, or epidural injection or implantation; topical administration; intratracheal; and intranasal administration. In some embodiments, the cord blood is administered systemically. In further embodiments, the cord blood is administered by intravenous injection.

### EXAMPLES

### Example 1: Phase I/II Trial to Determine the Effect Size of Change in GMFM-66 Score in Subjects Treated with hCT-MSC Compared to Allogeneic CB

### Overview

This study is a phase I/II, prospective, randomized, open-label trial designed to determine the effect size of change in GMFM-66 score in subjects treated with hCT-MSC or allogeneic CB and assess the safety of repeated doses of hCT-MSC in children with cerebral palsy. Children ages 2-5 years with cerebral palsy due to hypoxic ischemic encephalopathy, stroke, or periventricular leukomalacia may be eligible to participate. All participants will ultimately be treated with an allogeneic cell product at some point during the study. Participants will be randomized to one of three arms: (1) the “AlloCB” arm will receive one allogeneic CB infusion at the baseline visit; (2) the “MSC” arm will receive three hCT-MSC infusions, one each at baseline, three months, and six months; (3) the “natural history” arm will not receive an infusion at baseline but will receive an allogeneic CB infusion at 12 months. Motor outcome measures will be assessed at baseline, six-months, and one-year time points. Safety will be evaluated at each infusion visit and remotely for an additional 12 months after the final visit. Duration of study participation will be 24 months from the time of baseline visit. Randomization to treatment arms will be stratified by age and GMFCS level at study entry.

The primary endpoint is the difference between a participant's observed and expected changes in GMFM-66 score 12 months after the initial study infusion. Interval estimates will be reported separately for the hCT-MSC, AlloCB, and Natural History arms. Expected GMFM-66 scores at 12 months will be calculated based on the participant's baseline age, GMFCS level, and GMFM-66 score at study entry using published reference percentiles (Hanna et al., Phys Ther. 2008, 88(5):596-607).

### Purpose

The main purpose of this study is to estimate change in motor function 12 months after treatment with a single dose of allogeneic umbilical cord blood (AlloCB) or repeated doses of umbilical cord tissue-derived mesenchymal stromal cells (hCT-MSC) in children with cerebral palsy. In addition, this study will contribute much needed data to the clinical trials community on the natural history of the motor function in CP over shortterm (less than 1 year) time periods relevant to the conduct of clinical trials and assess the safety of AlloCB and hCT-MSC infusion in children with cerebral palsy.

Source of Unrelated CB Units for this Trial

The Carolinas Cord Blood Bank (CCBB) is one of the largest public cord blood banks in the nation. Established in 1998 with support from the National Heart and Blood Institute of the NIH, the CCBB has over 35,000 CB units in inventory and has distributed over 2,500 CB units for transplant to date. In 2012 the CCBB received approval from the FDA for its BLA application to market DUCORD, a stem cell product derived from umbilical cord blood, for use in transplants between unrelated donors and recipients. DUCORD is approved for use in hematopoietic stem cell reconstitution for patients with disorders affecting the hematopoietic system that are inherited, acquired, or result from myeloablative treatment. The CCBB currently collects from 10 hospital sites (8 in North Carolina, 1 in Atlanta, Ga. and 1 in Boston, Mass.). It also accepts CB donations from mothers delivering in any hospital in North Carolina and Atlanta through a kit donation program.

### Specifications for Qualification CB Units

Based on established criteria utilizing allogeneic CB for hematopoietic stem cell transplantation and our experience in treating more than 600 children with autologous CB for neurological conditions, we have established the following criteria to qualify banked CB units for cell therapy studies. All CB units utilized for this current study will be obtained from the Carolinas Cord Blood Bank. The CB unit must have:
1. Pre-cryopreservation total nucleated cell count (TNCC) documented and at least 10×10⁷/kg
2. Pre-cryopreservation viability ≥85% of total cells and ≥70% of CD34+ cells and a viable CD34 cell content ≥1.25×10⁶ cells
3. Pre-cryopreservation sterility culture performed and negative
4. Maternal infectious disease screening as follows: Testing must include negative results for Hepatitis B, Hepatitis C, HIV, HTLV, and syphilis. Additional screening, which is dependent on the timing of the CB collection, may be performed based on local and national regulations. Units from mothers who have a positive CMV antibody screen may be used as long as the CBV DNA on the cord blood plasma is negative
5. Test sample available for identity confirmation and potency testing
6. HLA typing performed and meets study-specific parameters
7. CD45+ viability ≥40% and CD34+ viability ≥70% on thawed test sample
   Source of MSCs for this Study: hCT-MSC

hCT-MSC is a third party MSC product manufactured from allogeneic donor digested umbilical cord tissue that is expanded for two passages in culture, cryopreserved, stored in the vapor phase of liquid nitrogen, and banked. The umbilical cord tissue is donated by healthy mothers delivering healthy full term babies after a normal pregnancy with written informed consent. The cells are manufactured, cryopreserved and stored in the Robertson GMP laboratory in the Marcus Center for Cellular Cures (MC3) (Duke University, Durham, N.C.).

Umbilical cord tissue is an attractive source of MSCs as it is readily available and easily obtained without consequence to the donor, is non-controversial, has a higher proliferative potential than MSCs from other postnatal sources (Drela et al., Cytotherapy. 2016, 18(4):497-509). Numerous preclinical studies have not demonstrated any evidence of tumorigenicity or toxicity of cord tissue derived MSCs (Park et al. Toxicol Res. 2016, 32(3):251-258). In early phase clinical trials published in English that utilized cord tissue-derived MSCs, in these 36 studies, including 695 patients and at least 1,416 doses of cord tissue-derived MSCs with follow-up ranging from three months up to six years, no severe adverse events were reported. Several more clinical trials of cord-tissue derived MSCs in various disease conditions are underway (clinicaltrials.gov).

### Study Rationale and Hypotheses

Previous studies suggest that adequately dosed autologous CB infusion can improve motor function in children with cerebral palsy. As it is not feasible that every child with cerebral palsy will have access to their autologous CB, this study will assess efficacy of two allogeneic sources of cells that can be available to all patients in need. The major goal of this study is to investigate change in motor function 12 months after treatment with two allogeneic cell sources, allogeneic CB and hCT-MSCs.

This study will generate important data regarding the effect size of change in motor function of these two cell sources and a natural history cohort to aid in the planning of future trials. The rationale for the study and for the potential benefit of cell therapy in cerebral palsy is based upon the following hypotheses:
We have demonstrated safety and dose-dependent efficacy of autologous CB infusions in children with cerebral palsy.
It is possible that different cell types, e.g. cord blood mononuclear cells versus cord tissue MSCs, may influence brain connectivity by different mechanisms.
Multiple doses of cells may be superior to a single dose of cells.
The developing brain exhibits remarkable plasticity, making young children ideal candidates for deriving maximal therapeutic benefit from restorative therapies, including CB.
CB cells, acting through paracrine mechanisms, may facilitate endogenous repair mechanisms and promote formation of new neural connections the motor cortex resulting in significant clinical improvements.
Brain connectivity plays an important role in the pathophysiology, and potentially mechanism of repair, of brain injury in children with cerebral palsy. Specifically, we hypothesize that (1) impairments in brain connectivity account for the motor deficits in children with cerebral palsy, (2) increases in brain connectivity have a direct impact on functional improvements, (3) children with cerebral palsy who receive CB infusions will exhibit greater increases in brain connectivity than children who receive placebo infusions, and (4) the severity of baseline brain connectivity abnormalities predict the potential for benefit of CB therapy.

### Study Design

This study is a phase I/II, prospective, randomized, open-label trial designed to assess the effect size of change in GMFM-66 score in subjects treated with hCT-MSC or allogeneic CB and assess the safety of repeated doses of hCT-MSC in young children with cerebral palsy. Children ages 2-5 years with cerebral palsy due to hypoxic ischemic encephalopathy, stroke, or periventricular leukomalacia may be eligible to participate. All participants will ultimately be treated with an allogeneic cell product at some point during the study. Participants will be randomized to one of three arms: (1) the “AlloCB” arm will receive one allogeneic CB infusion at the baseline visit; (2) the “MSC” arm will receive three hCT-MSC infusions, one each at baseline, three months, and six months; (3) the “natural history” arm will not receive an infusion at baseline but will receive an allogeneic CB infusion at 12 months. All participants will have an initial clinical evaluation to verify and classify the diagnosis of cerebral palsy and determine eligibility. They will return for study visits an additional two (AlloCB and natural history arms) or three (MSC arm) times. Outcome measures will be assessed at baseline, six-months, and one-year time points. Additional safety endpoints will be assessed remotely for 12 months after the final in-person visit.

### Study Objectives

Primary Objective: To determine the effect size of change in GMFM-66 score in children with cerebral palsy treated with a single dose of 10×107 cells/kg of allogeneic CB or three doses of 2×106 cells/kg of hCT-MSC.

Secondary Objective: To assess the safety of repeated doses of hCT-MSC in children with cerebral palsy.

Exploratory Objectives: (1) To determine the change in the Peabody Developmental Motor Scale-2 (PDMS-2) score at 6 and 12 months in children treated with allogeneic CB or hCT-MSC. (2) To analyze the change in normalized total brain connectivity, as measured by brain MRI with DTI, from baseline to 12 months. (3) To assess changes functional and quality of life measures at 6 and 12 months.

### Study Design—General Design

This study is a phase I/II, prospective, randomized, open-label trial designed to determine the effect size of change in GMFM-66 score in subjects treated with hCTMSC or allogeneic CB and assess the safety of repeated doses of hCT-MSC in children with cerebral palsy. Children ages 2-5 years with cerebral palsy due to hypoxic ischemic encephalopathy, stroke, or periventricular leukomalacia may be eligible to participate.

All participants will ultimately be treated with an allogeneic cell product at some point during the study. Participants will be randomized to one of three arms: (1) the “AlloCB” arm will receive one allogeneic CB infusion at the baseline visit; (2) the “MSC” arm will receive three hCT-MSC infusions, one each at baseline, three months, and six months; (3) the “natural history” arm will not receive an infusion at baseline but will receive an allogeneic CB infusion at 12 months. Motor outcome measures will be assessed at baseline, six-months, and one-year time points. Safety will be evaluated at each infusion visit and remotely for an additional 12 months after the final visit. Duration of study participation will be 24 months from the time of baseline visit. Randomization to treatment arms will be stratified by age and GMFCS level at study entry.

### Study Design—Study Endpoints

Primary Endpoint: The primary endpoint is the difference between a participant's observed and expected changes in GMFM-66 score 12 months after the initial study infusion. Interval estimates will be reported separately for the hCT-MSC, AlloCB, and Natural History arms. Expected GMFM-66 scores at 12 months will be calculated based on the participant's baseline age, GMFCS level, and GMFM-66 score at study entry using published reference percentiles (Hanna et al. Phys Ther. 2008, 88(5):596-607).

Secondary Endpoints: The secondary endpoint is the number of adverse events occurring over the 12-month period post-infusion with hCT-MSC or AlloCB.

Exploratory Analyses:
Observed GMFM-66 score at baseline, 6, and 12 months
Change in the Peabody Developmental Motor Scale-2 (PDMS-2) score at 6 and 12 months.
Change in normalized total brain connectivity, as measured by brain MRI with DTI, from baseline to 12 months.
Change in functional and quality of life measures at 6 and 12 months.

### Research Participant Selection and Withdrawal—Study Population

Ninety children ages 2-5 years with spastic cerebral palsy.

### Research Participant Selection and Withdrawal—Inclusion Criteria

1. Age ≥24 months and ≤60 months adjusted age at the time of enrollment. Patient age will be adjusted for prematurity if the patient was born at <37 weeks gestation.
2. Diagnosis: Unilateral or bilateral spastic cerebral palsy secondary to in utero or perinatal stroke/hemorrhage, hypoxic ischemic encephalopathy (including, but not limited to, birth asphyxia), and/or periventricular leukomalacia.
3. Performance status: Gross Motor Function Classification Score levels I-IV
4. Review of brain imaging (obtained as standard of care prior to study entry) does not suggest a genetic condition or brain malformation.
5. Legal authorized representative consent.

### Research Participant Selection and Withdrawal—Exclusion Criteria

1. Available qualified autologous cord blood unit.
2. Hypotonic or ataxic cerebral palsy without spasticity.
3. Autism and autistic spectrum disorders without motor disability.
4. Hypsarrhythmia.
5. Intractable seizures causing epileptic encephalopathy.
6. Evidence of a progressive neurologic disease.
7. Has an active, uncontrolled systemic infection or documentation of HIV+ status.
8. Known genetic disease or phenotypic evidence of a genetic disease on physical exam.
9. Concurrent genetic or acquired disease or comorbidity(ies) that could require a future allogeneic stem cell transplant.
10. Requires ventilatory support, including home ventilator, CPAP, BiPAP, or supplemental oxygen.
11. Impaired renal or liver function as determined by serum creatinine >1.5 mg/dL and/or total bilirubin >1.3 mg/dL except in patients with known Gilbert's disease.
12. Possible immunosuppression, defined as WBC <3,000 cells/mL or absolute lymphocyte count (ALC) <1500 with abnormal T-cell subsets.
13. Patient's medical condition does not permit safe travel.
14. Previously received any form of cellular therapy.

### Research Participant Selection and Withdrawal—Research Participant Recruitment and Screening

Patients may be recruited through IRB-approved advertising for the study on the websites of CB banks, parent sponsored websites, the NMDP website, selected cerebral palsy societies, local medical providers, and through a record of inquiries for previous studies (brain injury database. Separate IRB approval will be obtained for any advertisements.

Screening for this study is conducted under a separate, IRB-approved screening protocol (Pro00063563). Under this protocol, after written informed consent is obtained from a parent/guardian, the patient's medical records, videos, and results of brain imaging are obtained and reviewed. The medical review is conducted by a team of pediatric nurses, nurse practitioners, and physicians to identify the presence of any exclusion criteria. If no exclusion criteria are identified, screening labs are performed and a search may be conducted to identify a suitably matched CB unit.

### Study Products—Allogeneic Umbilical Cord Blood

Allogeneic unrelated donor CB units utilized for this trial will be obtained from the Carolinas Cord Blood Bank, an FDA licensed Public Cord Blood Bank at Duke University Medical Center. CB donors must be eligible for donation to a public cord blood bank for allogeneic use. Donor eligibility screening via questionnaires is performed in accordance with CFR 1271.75 and infectious disease testing is performed in accordance with CFR 1271.80 and 1271.85. The unit must also have an appropriate degree of HLA matching and meet product specifications as detailed below.

All potential study participants will undergo high resolution HLA typing at HLA-A, B, and HLA-DRB1 via blood or buccal swab. Patients receiving allogeneic CB will have HLA typing performed on two separate samples for confirmation. Allogeneic units that are potential matches will initially be identified from a search of the Carolinas Cord Blood Bank. The best available HLA-matched (≥4/6), using intermediate level matching at HLA Class I A and B and high resolution-allele level matching at HLA Class II, DRB1, CB unit with a pre-cryopreservation nucleated cell dose ≥12×10⁷ cells/kg will be selected. Once a unit is selected, HLA typing will be used to confirm the original HLA typing and to select the best matching unit. When possible, at least 1 match at each HLA loci will be prioritized. A CB unit must be at least 4/6 HLA-matched with the patient.

Recipients' ABO/Rh blood typing will be obtained. CB units will not be selected based on ABO typing. However, an Rh negative CB unit will be selected for Rh negative female participants to avoid Rh sensitization in young females.

Results of initial testing at the cord blood bank must include a pre-cryopreservation TNCC, viability and sterility culture. Pre-cryopreservation TNCC must be ≥12×10⁷/kg to target administration of 10×10⁷ cells/kg post thaw, sterility cultures must have been negative, total viability must have been ≥85%, and CD34+ cell viability must have been ≥70%.

A test vial or segment must be available from each CB unit for potency testing and confirmatory HLA typing. The segment will be detached from the candidate unit and tested for potency and identity (HLA-confirmatory typing) per Standard Operating Procedures in the CCBB at Duke. Units will be deemed acceptable for the trial if viability of the CD45 cell population is ≥40% and viability of the CD34 cell population is ≥70%. CFU growth, expression of aldehydehydrogenase and CD34 will be described but will not be a specification for study enrollment.

Prior to the patients' arrival, their designated CB unit will be transferred from the Carolinas Cord Blood Bank to the Duke STCL, located in the same building, where it will be stored in a liquid nitrogen freezer until the day of infusion. On the infusion day, the CB will be thawed and washed in dextran/albumin and resuspended in an appropriate volume based on recipient weight for administration to the patient the standard fashion (Rubinstein et al. Proc Natl Acad Sci USA. 1995, 92(22):10119-10122) per SOP STCL-PROC-036. At the time of thawing, standard studies listed (see Table 1) will be performed. Only TNCC is utilized for release. A maximum dose of 10×10⁷ TNC/kg will be prepared for infusion in a syringe or bag and infused over 2-25 minutes.

TABLE 1
Post-Thaw Cord Blood Unit Testing

| Test | Specifications |
|---|---|
| Total Nucleated Cell Count (TNCC) | Report; used to calculate |
| | final dose |
| Viability | Report |
| Viability of the CD34+ population* | ≥70% |
| Viability of the CD45+ population* | ≥40% |
| Sterility** | No Growth |
| Colony Forming Unit (CFU) growth | Report |
| ALDH^{br} as a percentage of CD45+ cells | Report |

*Viability of the CD34+ and CD45+ cells post-thaw was previously tested on a segment and required to meet the specification of ≥70%. Therefore, for the clinical product, we will report but not use the post thaw viability as a release criteria.
**If a positive culture is obtained after product administration, a plan is put into effect to notify the clinical and study teams and treat the patient if indicated.

Study Products—Human Umbilical Cord Tissue-Derived Mesenchymal Stromal Cells (hCT-MSC)

hCT-MSCs are manufactured under cGMP in a clean room ISO 7 facility and are a product of allogeneic cells manufactured from digested umbilical cord tissue that is expanded in culture, cryopreserved and banked. hCT-MSCs are manufactured in the Duke CT2 GMP cell manufacturing lab from umbilical cord tissue harvested from the placenta from normal term deliveries where the baby's cord blood was donated to the Carolinas Cord Blood Bank, an FDA-licensed, FACT-accredited, public cord blood bank at Duke University Medical Center, after written informed consent from the donor baby's mother. Cord tissue is harvested from the placentas of male babies delivered by elective C-section after a normal, full-term pregnancy. Donor screening questionnaires are completed by the maternal donor, and maternal blood is tested for communicable diseases by the CLIA-certified donor screening laboratory at the American Red Cross in Charlotte, N.C. Donors must be eligible for donation to a public cord blood bank for allogeneic use. After delivery of the placenta and cord, the cord blood is aseptically drained from the placenta. Then the cord is dried and cleaned with chloropreps, separated from the base of the placenta, placed in a sterile bottle containing Plasmalyte A, and transported to the Robertson Clinical and Translational Cell Therapy CT2 GMP cell processing laboratory at room temperature in a validated container.

In the clean room manufacturing suite, in a biosafety cabinet, the cord tissue is removed from the media, placed in sterile dishes, cut into small pieces and then minced and digested in the Miltenyi Biotec GentleMacs Octo Dissociator with GMP-grade enzymes: hyaluronidase, DNase, collagenase, papain. The resultant cell suspension is placed in culture in Prime XV MSC Expansion XSFM (Irvine Scientific) media with 1% platelet lysate and grown to confluence (˜7-14 days) to establish the P0 culture. To establish the master cell bank, P0 is harvested and cryopreserved in cryovials with Cryostor 10 media (BioLife), and stored in the vapor phase of liquid nitrogen. P1 and P2 cultures are grown under similar conditions, in HYPERFlasks or HYPERStacks without platelet lysate, as needed to create the working cell bank and product for administration, respectively. Cells from P1 and P2 are removed from plastic cultureware using TrypLE (Gibco). The final product is derived from the P2 cultures which are harvested into plasmalyte with 5% human serum albumin, washed and cryopreserved in compartment cryobags containing 50-100 million cells in a final concentration of 10% DMSO with dextran (Akron Scientific). On the day of administration, one compartment is thawed, diluted in 10-40 mLs of plasmalyte IV solution, placed in a syringe or bag and transported to the bedside for administration over 30-60 minutes.

At each passage, the cell product is characterized by assessing cell surface phenotype by flow cytometry and functional assays via T-cell proliferation and organotypic models of microglial activation. Each lot, prior to cryopreservation of P2, will also be tested for sterility, endotoxin and mycoplasma and these tests must meet specifications. For dosing, release testing after thaw and dilution will include TNCC and viability via cellometer. Patients will be dosed with 2×106 hCT-MSCs/kg based on the post thaw count.

Process and Final Formulation

hCT-MSC is manufactured from a single umbilical cord tissue in a series of three steps that generate a master cell bank, a working cell bank, and the study product. The product for each step is cryopreserved in a controlled rate freezer and stored in the vapor phase of liquid nitrogen. At P2, a representative cryobag is thawed and qualified prior to the treatment of any patients with that lot of product. Testing for product release includes total nucleated cell count, viability, phenotype, functional assays, endotoxin, mycoplasma, gram stain and sterility. Each lot of cells is also tested for adventitial viruses prior to cryopreservation.

On the day of treatment, cells are thawed per SOP STCLAOP-028 JA2 and then diluted in 10-40 mLs of plasmalyte A+5% human serum albumin (HSA). An aliquot is removed for cell count, viability, and sterility culture. If the cells are ≥70% viable, the final product volume is adjusted to deliver 2×10⁶ cells/kg to the study subject. The cells are delivered to the bedside in a syringe containing plasmalyte A, 5% HSA, and residual DMSO. Any removed cell suspension is inoculated into aerobic and anaerobic culture bottles for sterility testing. The cells have a four-hour expiry at room temperature post thaw.

The hCT-MSC final product will be released conditionally for administration to the patient after testing a post thaw cell count and viability. Final release will occur after the 14-day sterility culture period for the study product. In the event that a sterility culture turns positive after administration of the product, the organism will be identified and antibiotic sensitivities performed. The patient's family will be contacted to determine if they are symptomatic (i.e. fever or other signs of infection). Asymptomatic patients will be observed but will not be treated with antibiotics. Symptomatic patients will be evaluated and treated accordingly, with blood cultures and antibiotics as appropriate. All patients receiving a product with subsequent positive sterility test will be followed with daily contact by a study nurse for 14 days after the positive sterility test is noted.

Further manufacturing and testing details may be found in the U.S. Provisional Application to Kurtzberg et al. entitled “Methods for the Treatment of Autism Spectrum Disorders Using Human Umbilical Cord Tissue-Derived Mesenchymal Stromal Cells,” filed concurrently herewith, the contents of which are hereby incorporated by reference in their entirety.

Study Products—Donor Screening for CB and hCT-MSC

Donor screening and testing is performed per Carolinas Cord Blood Bank standard operating procedures to meet all requirements in 21CFR Part 1271. The screening and testing is current with recommendations and is approved by the FDA under biological license number 1870. Maternal donors of umbilical cord blood are screened and tested for HIV-1, HIV-2, HIV-O, hepatitis B virus (HBV, surface antigen and core antibody), hepatitis C virus (HCV) antibody, Treponema pallidum (syphilis), Creutzfelds-Jakob Disease (CJD, screening only), Chagas Disease, human T-lymphotropic virus types 1 and 2 (HTLV-1, HTLV-2) and total antibodies against CMV. Nucleic acid testing for HIV-1/2/O, HBV, West Nile Virus and HCV are also performed on maternal blood. Screening for Zika virus may also performed.

Because the cord tissue used for this study will be obtained from donors consented for cord blood donation to the Carolinas Cord Blood Bank, they will undergo donor screening and infectious disease testing per Carolinas Cord Blood Bank standard operating procedures. The cord blood-associated maternal samples and cord tissue MSC samples will be retained as reference samples for future testing as part of this study.

### Study Products—Packaging of Study Products

All cellular products receive a unique identification number (ISBT Demand 128 bar code) to ensure product integrity and maintain chain of custody. The clinical site or cord blood bank assigns an ISBT Demand 128 bar code label to the CB unit or hCT-MSC product, which is placed on the product bag/syringe directly or via tie tag. Products are transported from the STCL to the infusion site in a validated cooler by a trained courier.

### Study Products—Administration of Study Product

Patients will arrive in clinic on the morning of their scheduled infusion. A peripheral IV will be placed either by an anesthesiologist, clinical staff or study staff and premedication with Benadryl 0.5 mg/kg/dose IV and Solumedrol 0.5-1 mg/kg IV will be administered. Allogeneic CB products will be administered intravenously over 5 to 25 minutes under direct physician supervision. hCT-MSC products will be administered intravenously over 30-60 minutes under direct supervision. Vital signs (heart rate, blood pressure, temperature, respiratory rate) will be checked upon arrival to the clinic and as clinically indicated. Pulse oximetry will be monitored continuously throughout the infusion and for at least 5 minutes post infusion. Patients will be hydrated with standard intravenous fluids as tolerated and observed for at least one hour post infusion.

### Study Plan—Overview

Parents/Guardians who have previously contacted our program and have a child who may meet eligibility criteria for this study will be notified that this study is available. After initial contact, parents/guardians of potential research participants will have an initial phone interview with study personnel to describe the study, verify basic eligibility criteria, and confirm their interest in participation. The participant's eligibility will then be screened through review of medical records, video, laboratory testing, and imaging under a separate screening protocol.

Once all screening is complete and the patient is likely to meet study criteria, a suitable unrelated donor CB unit will be identified at the Carolinas Cord Blood Bank. The CB unit will be screened as described in section 6. Participants will then travel to Duke for their first visit. On day 1, written informed consent will be obtained. Patient eligibility will be confirmed by a physical observation and verification of cerebral palsy diagnosis and GMFCS level. If no exclusion criteria are realized, the participant will be randomized to a treatment arm. During their first visit, all participants will have physical therapy evaluations, and a subset of patients will undergo brain MRI. Participants will have study infusions as determined by their assigned treatment arm (at baseline only for AlloCB; at 12-months only for Natural History; at baseline, 3-, and 6-months for MSCs).

Participants will be evaluated the day after each infusion, and parents will be contacted for phone follow-up ˜2 weeks after each infusion. All participants will return to Duke six (motor assessments) and 12 months (motor assessments and brain MRI) after the baseline visit. Participants on the MSC arm will also return at three months for an hCT-MSC infusion. A remote safety assessment will be performed via phone or email at 24 months post-infusion.

### Study Plan—Patient Screening

Initial patient screening will be conducted with informed consent under a separate protocol and will include a review of medical records, videos, and initial laboratory testing. If no exclusion criteria are identified, informed consent will be obtained over the phone, the patient will be randomized to treatment arm. If indicated (AlloCB and Natural History arms), an unrelated donor CB unit will be identified at the Carolinas Cord Blood Bank. Participants will travel to Duke for initial evaluation. Evaluations and treatments will be conducted in the outpatient setting. A physical exam and baseline GMFCS assessment will be conducted to confirm eligibility, and the participant undergo the remainder of the study evaluations.

### Study Plan—CB Unit Selection

For participants randomized to the AlloCB and Natural History arms, an allogeneic unrelated donor CB unit will be identified at the Carolinas Cord Blood Bank. HLA typing will be obtained on the patient, and the best available HLA-matched CB unit with a precryopreservation nucleated cell dose ≥12×10⁷ cells/kg will be chosen. When possible, at least 1 match at each HLA loci will be prioritized. An Rh negative CB unit will be selected for Rh negative female participants to avoid Rh sensitization in young females.

Once a suitable allogeneic CB unit has been deemed an acceptable match, a sample of the CB unit will be tested for potency in the Duke STCL. If results of these tests are satisfactory, the CB unit will be delivered to the Duke STCL in the frozen state.

### Study Plan—Study Product Infusion

On the day of infusion, CB cells or hCT-MSC product will be prepared by the STCL and provided for infusion of the patient in the outpatient clinic under the supervision of the study team and Pediatric Blood and Marrow Transplant Program staff. A peripheral IV will be placed by clinical staff, anesthesia or a member of the study team. Prior to the study infusion, premedications (Benadryl and Solumedrol) will be administered. CB cells will have a four-hour expiry at room temperature post-thaw.

Allo CB infusion will be given over approximately 5-25 minutes and hCT-MSC infusions over 30-60 minutes using standard practices. The child will receive 1-1.5× maintenance IV fluids as described below and be observed in the clinic for a minimum of one hour after the infusion. Patients will be discharged from clinic after at least one hour providing all vital signs are at their baseline and they are awake and asymptomatic with no evidence of toxicity. Patients will be evaluated by study staff the day after the infusion to assess for any infusion-related adverse reactions or complications. A phone call to parents/guardians by study staff to assess safety of the infusion will be conducted two weeks after the infusion.

Maintenance IV Fluid Rate (Holliday-Segar
Method from Harriet Lane Handbook)

| Body weight | mL/kg per day | |
|---|---|---|
| 1st 10 kg | 100 | divided by 24 hr/day |
| 2nd 10 kg | 50 | divided by 24 hr/day |
| each add'l kg | 20 | divided by 24 hr/day |

If a patient has evidence of illness on the day of planned infusion, including but not limited to fever >38.5° C., vomiting, diarrhea, or respiratory distress, the infusion will be postponed.

### Study Plan—Care During Unexpected Events

In the event that a patient develops signs or symptoms of anaphylaxis including urticaria, difficulty breathing, cough, wheezing, or vomiting during their CB infusion, the infusion will be terminated and appropriate medical therapy initiated.

### Study Plan—Motor Assessments

Gross Motor Function Measurement-66 (GMFM-66): The GMFM-66 is a standardized observational instrument designed and validated to measure change in gross motor function over time in children with cerebral palsy. Developmental curves of expected progression have been published for children ages 2-12 years (Hanna et al. Phys Ther. 2008, 88(5):596-607; Rosenbaum et al. Jama. 2002, 288(11):1357-1363) allowing for the calculation of future expected scores based on the baseline age, GMFCS level, and GMFM-66 score. The GMFM-66 consists of 66 items, divided into five categories: lying and rolling, sitting, crawling and kneeling, standing, and walking, running, and jumping.

Each item is scored on a four-point Likert scale. The GMFM-66 is a subset of the GMFM-88, which contains an additional 22 items, primarily in the lying and rolling category. Both measures have been validated in children with cerebral palsy from 5 months to 16 years of age. A 5-year old child without motor disabilities is able to reach the maximum score (Russell et al. Gross Motor Function Measure (GMFM-66 & GMFM-88) User's Manual. London: Mac Keith Press; 2013). A computer program, the Gross Motor Ability Estimator, is used to calculate the GMFM-66 total scores. The primary endpoint of this study is the difference between a child's actual and expected changes in GMFM-66 score 12 months after the initial study infusion. Control (placebo) and treated patients will be compared.

When possible, the entire GMFM-88 will be performed, and subsets may be analyzed as exploratory endpoints.

Peabody Developmental Motor Scales (PDMS-2): The PDMS-II is a standardized assessment of early childhood motor development that evaluates both gross and fine motor skills. It is designed for children from birth through 5 years of age. The assessment is composed of six subtests that measure interrelated motor abilities that develop early in life (i.e., reflexes, stationary, locomotion, object manipulation, grasping, and visual-motor integration). Gross Motor Quotient, Fine Motor Quotient, and Total Motor Quotient composite scores are obtained. For this study, the Gross Motor Quotient will be obtained and analyzed as a secondary endpoint.

### Study Plan—Functional and Quality of Life Assessments

Pediatric Evaluation of Disability Inventory-Computer Adaptive Test (PEDI-CAT): The PEDI-CAT measures abilities in three functional domains: Daily Activities, Mobility, and Social/Cognitive. The computerized adaptive version is intended to provide an accurate and precise assessment of a child's abilities while increasing efficiency and reducing respondent burden by utilizing item response theory statistical models to determine which items are assessed within each domain based on responses to prior items.

Pediatric Quality of Life Inventory 4.0, Generic Core Scale and Cerebral Palsy Module (PedsQL) (Varni et al. Developmental medicine and child neurology. 2006, 48(6):442-449). The PedsQL General Core Scales and Cerebral Palsy Module are composed of parallel child self-report and parent proxy-report formats. The 35-item PedsQL Cerebral Palsy Module encompasses seven scales and generates a standard score: (1) Daily Activities (9 items), (2) School Activities (4 items), (3) Movement and Balance (5 items), (4) Pain and Hurt (4 items), (5) Fatigue (4 items), (6) Eating Activities (5 items), and (7) Speech and Communication (4 items).

### Study Plan—Imaging Assessments

Participants' brain imaging obtained previously as standard of care will be reviewed by a member of the Brain Imaging Analysis Center (BIAC) team to determine if accurate anatomical image parcellation would be likely on a brain MRI. Those participants for whom usable data is likely to be obtained (estimated as approximately two-thirds of eligible participants) will undergo brain MRI with diffusion tensor imaging (DTI). Diffusion weighted images will be acquired on a 3 Tesla GE scanner (Waukesha, Wis.). T1-weighted images will be obtained with an inversion-prepared 3D fast spoiled-gradientrecalled (FSPGR) pulse sequence. These images will be analyzed to obtain measures of whole brain connectivity.

### Statistical Considerations—Study Design

This study is a phase I/II, prospective, randomized, open-label trial designed to provide interval estimates of the 12-month change in motor function after treatment with AlloCB and hCT-MSC, provide additional data to the clinical trials community on the natural history of the motor function in CP over short-term (less than 1 year) time periods relevant to conduct of clinical trials, and assess the safety of repeated doses of hCT-MSC and a single dose of AlloCB in children with cerebral palsy.

Children ages 2-5 years with cerebral palsy due to hypoxic ischemic encephalopathy, stroke, or periventricular leukomalacia will be eligible to participate. All participants will ultimately be treated with an allogeneic cell product at some point during the study. Participants will be randomized (1:1:1) to one of three arms: (1) the “AlloCB” arm will receive one allogeneic CB infusion at the baseline visit; (2) the “MSC” arm will receive three hCT-MSC infusions, one each at baseline, three months, and six months; the “natural history” arm will not receive an infusion at baseline but will receive an allogeneic CB infusion at 12 months. The occurrence of adverse events will be evaluated at 3, 6, 12, and 24 months post-randomization in all participants. Motor function outcome measures will be assessed at baseline, six-months, and one-year time points in all participants. Duration of study participation will be 24 months from the time of the baseline visit. Randomization will be stratified by age (2-3 years vs. 4-5 years) and GMFCS Level (I/II or III/IV).

### Statistical Considerations—Accrual

It is estimated that up to 8-12 research participants will be enrolled each month and that approximately 12-15 months of accrual will be necessary to enroll 90 participants.

### Statistical Considerations—Study Duration

Each subject's participation in the study will be 24 months, with clinic visits occurring during the first 12 months and a remote safety assessment at 24 months. Given that accrual will take up to 15 months it is estimated that the remote safety assessment will be conducted on that last patient 39 months (3.25 years) after the study opens.

### Statistical Considerations—Primary and Secondary Endpoints

The primary endpoint of this study is the difference between a child's observed and expected changes in GMFM-66 score 12 months after the initial study infusion. This study will provide separate interval estimates of the mean of this outcome measure in patients assigned to the hCT-MSC, AlloCB, and Natural History arms at 12-months. The secondary endpoint of this study is the number of adverse events occurring over a 12-month period post-treatment with hCT-MSC or AlloCB.

### Statistical Considerations—Sample Size and Power Calculations

The sample size for this study was selected to provide a high level of precision for estimating the mean of the observed minus expected 12-month change on the GMFM-66 in each of the study arms, and to provide a high probability of detecting commonly occurring adverse events after infusion with AlloCB or hCT-MSC.

As shown in Table 2 below, a sample size of 30 patients per group provides a 95.8% probability of detecting common adverse events that occur in 10% of infusions (with hCT-MSC or AlloCB). This sample size also provides a 78.5% probability of observing events that occur in 5% of infusions, and a 26.0% probability of observing rare events that occur in 1% of infusions.

TABLE 2
Probability of Observing One or More Events
with Various Sample Sizes*

| | |
|---|---|
| True Probability | Probability (%)* |

| of an Event (%) | N = 20 | N = 30 | N = 40 | N = 50 |
|---|---|---|---|---|
| 1 | 18.25 | 26.0 | 33.1 | 39.5 |
| 5 | 64.2 | 78.5 | 87.1 | 92.3 |
| 10 | 87.8 | 95.8 | 98.5 | 99.5 |
| 20 | 98.8 | 99.9 | 100.0 | 100.0 |
| 50 | 100.0 | 100.0 | 100.0 | 100.0 |

*Binomial probability of 1 or more independent events.

The sample size for this study must also support estimation of the mean observed minus-expected GMFM-66 change score at 12 months post-intervention with MSC, AlloCB, and in the Natural History arm. Thus, three interval estimates will be constructed using the t-distribution as follows.

$$( \overline{x} - {t}_{\frac{α}{2}} * \frac{s}{\sqrt{n}} , \overline{x} + {t}_{\frac{α}{2}} * \frac{s}{\sqrt{n}} )$$

The margin of error E is the confidence interval half-width:

$$E = {t}_{\frac{α}{2}} * \frac{s}{\sqrt{n}}$$

The margin of error for this study was selected as 2 points with a confidence level of 95%. The following formula was solved iteratively to obtain the sample size for each treatment group.

$$N = {( \frac{{t}_{\frac{α}{2}} * s}{E} )}^{2}$$

The standard deviation, s, was estimated using 36 participants in the CP-AC trial who met age and GMFCS inclusion criteria for the present study: 5.16 (95% CI: 4.18, 6.13). Starting with a sample size of 20, and assuming a standard deviation of 5.16, a total of 3 iterations were required to reach a final group sample size of 28 as shown in Table 3 below.

TABLE 3

| | | Degrees of | | |
|---|---|---|---|---|
| Iteration # | Starting N | Freedom | tα_{/2} | Ending N |
| 1 | 20 | 19 | 2.093 | 29 |
| 2 | 29 | 28 | 2.048 | 28 |
| 3 | 28 | 27 | 2.052 | 27 |

Therefore, a group size of 28 patients allows for 95% confidence in the estimation of the mean 12-month observed-minus-expected GMFM-66 change score in one of the study arms (Natural History, MSC or AlloCB) with a margin of error of no more than 2. This sample size is also concordant with what is required (N=30) for reasonable probability of detecting commonly occurring adverse events, as described above. Finally, if the standard deviation of the secondary outcome measure is as high as that indicated by the upper limit of the 95% confidence interval from the CP-AC study (6.13 points) then a sample of 126 patients allows for a margin of error no larger than ˜2.5 points for each of the three interval estimates.

The total sample size for this study is therefore set at 90 patients (30 per group).

### Statistical Considerations—Analysis Plan

Analysis Populations

The following populations are defined to support analyses of the primary and secondary endpoints.

Intention to Treat Population

This population will include all enrolled and randomized participants according to their assigned treatment. The primary endpoint will be evaluated in this population.

Safety Population

The safety population defines the patients in whom the secondary endpoint will be evaluated and will include all subjects who received at least 1 infusion. Analyses of the Safety Population will be conducted using an as-treated approach, which considers each patient according the treatment actually received rather than the treatment they were assigned.

Timing of Analyses

The analysis of the primary and secondary outcome measures will be conducted when the last patient reaches their 12-month visit. An update will be made to the safety analyses when the last patient reaches their 24-month visit.

Demographics, Baseline Characteristics, and Disposition

Demographics and baseline characteristics will be summarized for all research participants and separately by randomized assignment. Characteristics to be examined include age, sex, race/ethnicity, baseline GMFM-66 score, GMFCS level, and etiology of CP. The number of participants entering and completing the study will be diagrammed using the CONSORT guidelines.

Analysis of the Primary and Secondary Endpoints

The occurrence of adverse events in the Safety Population will be summarized descriptively in tables and figures for all subjects and separately by treatment received. Estimates of the mean observed-minus-expected GMFM-66 change score at 12 months will be reported in the Intention to Treat Population along with 95% confidence intervals as described above.

We claim:

1. A method of treating a patient with cerebral palsy comprising administering allogeneic cord blood at a dose between 7.5×10⁷ and 12.5×10⁷ total nucleated cells/kg.

2. The method of claim 1, wherein the dose is about 10×10⁷ total nucleated cells/kg.

3. The method of claim 1 wherein the dose is 10×10⁷ total nucleated cells/kg.

4. The method of claim 1, wherein the cord blood is administered systemically.

5. The method of claim 2, wherein the cord blood is administered systemically.

6. The method of claim 3, wherein the cord blood is administered systemically.

7. The method of claim 4, wherein the cord blood is administered by intravenous injection.

8. The method of claim 5, wherein the cord blood is administered by intravenous injection.

9. The method of claim 6, wherein the cord blood is administered by intravenous injection.

10. The method of claim 1, wherein the patient is a human.

11. The method of claim 2, wherein the patient is a human.

12. The method of claim 3, wherein the patient is a human.

13. The method of claim 10, wherein the human is a human child under 18 years of age.

14. The method of claim 11, wherein the human is a human child under 18 years of age.

15. The method of claim 12, wherein the human is a human child under 18 years of age.
